(19) United States
(12) Patent Application Publication
(10) Pub. No.: US 2017/0079866 A1
(43) Pub. Date: Mar. 23, 2017

(54) Personal Massage Apparatus

(71) Applicant: Tahina Policard, Greece, NY (US)

(72) Inventor: Tahina Policard, Greece, NY (US)

(21) Appl. No.: 15/272,274

(22) Filed: Sep. 21, 2016

Related U.S. Application Data
(60) Provisional application No. 62/221,927, filed on Sep. 22, 2015.

(51) Int. Cl. A61H 1/00; A61B 5/11; A61N 1/36; A61B 5/107

(52) U.S. Cl. CPC A61H 1/008; A61B 5/1075; A61B 5/11; A61N 1/36014; A61H 2201/5058; A61H 2205/081; A61H 2201/01; A61H 2201/10; A61H 2201/5043

(57) ABSTRACT

Disclosed are various embodiments for implementing and creating a customizable artificial hand massage apparatus configured to simulate actual hand movements associated with a human user. The customizable artificial hand massage apparatus may also be configured to execute a massage application. The massage application may be executed to store information associated with the user of the customizable artificial hand massage apparatus, detect sounds, detect motion, determine an amount of gas expelled from the user of the customizable artificial hand massage apparatus, determine the number of times an infant burps, detects how far an object or person is located with respect to the customizable artificial hand massage apparatus, and transfer information to a separate control application.

### POLICARD

This application claims the benefit of U.S Provisional Application No. 62221927 filed on Sep. 22, 2015 the contents of which are herein incorporated by reference in its entirety.

### BACKGROUND

Adults often experience stress and tension in their bodies that may sometimes result in headaches, back and neck pain, and other problems. Consequently, people are looking to massage for much more than just relaxation. Massage therapy can be effective for a variety of conditions for adults, including arthritis, lower back pain, insomnia, headaches, anxiety, circulatory problems, and recovery from a sports injury.

Massage therapy can also be effective for soothing infants. Babies often exhibit uncontrollable crying due to pain or discomfort that may sometimes be caused by gas, a growing digestive system, hormones that cause stomach pain, oversensitivity, over simulation, or a developing nervous system. Challenges exist for parents of fussy babies to help to stop the infants from crying and to relieve any pain, discomfort that the infant may be experiencing.

### BRIEF DESCRIPTION OF THE DRAWINGS

FIG. 1. FIG. 1 shows an example of a customizable artificial hand massage apparatus according to various embodiments of the present disclosure.

FIG. 2. FIG. 2 shows a networked environment according to various embodiments of the present disclosure.

FIG. 3. FIG. 3 shows drawing of one example of a server in the networked environment of FIG. 1 according to various embodiments of the present disclosure.

### DETAILED DESCRIPTION

The present disclosure relates to the implementation and creation of a customizable artificial hand massage apparatus configured to simulate actual hand movements associated with a human user. The customizable artificial hand massage apparatus may be configured to mimic the actual massaging movements of a human user's hands and fingers. The customizable artificial hand massage apparatus may also be customized such that the artificial hand has the equivalent dimensions associated with the hand of a particular human user. Additionally, the customizable artificial hand may be configured for use in a variety of products such as, for example, a garment, a book bag a baby carrier, a baby car seat, a baby stroller, a built in automobile seat, a baby swing, a mattress, a blanket, a sheet, a rocker, a high chair, a pillow and other products. Alternatively, the customizable artificial hand apparatus may be configured to operate as a stand-alone device. The customizable artificial hand massage apparatus may also be configured to execute a massage application. The massage application may be executed to store information associated with the user of the customizable artificial hand massage apparatus, detect sounds, detect motion, determine an amount of gas expelled from the user of the customizable artificial hand massage apparatus, determine the number of times an infant burps, detects how far an object or person is located with respect to the customizable artificial hand massage apparatus, and transfer information to a separate control application.

The control application may be configured for use on a client device such as, for example, a laptop or desktop computer system, a smartphone, a tablet, or any other type of client device. The control application may be used to monitor and control one or more settings associated with the customizable artificial hand massage apparatus. Settings that may be monitored or controlled using the massage control application include the mode of operation, velocity, speed, strength, pressure, and other settings associated with the massage apparatus.

Additionally, the customizable artificial hand massage apparatus may be configured to operate in different modes and may have different settings for adults and infants. Modes of operation of the customizable artificial hand massage apparatus may include for example, Swedish massage, deep tissue massage, shiatsu, prenatal massage, burping, soothing, circulation massage, patting, fingertip tapping, rubbing, stroking, karate chop, rolling, pulsing, percussion, kneading, beating, and other modes that duplicate hand movements. For example, the customizable artificial hand massage apparatus may be configure to determine when an amount tension is relieved from muscles and be configured to automatically turn off or automatically adjust the amount of pressure applied to relieve the tension based in at least part on the amount of tension detected in the muscles.

In order to perform the massage function, the customizable artificial hand massage apparatus may be include electrical stimulation devices, such as, for example, transcutaneous electrical nerve stimulation unit (TENS), neuromuscular electrical stimulation devices (NMES), galvanic stimulation devices, interferential therapy, microcurrent stimulation device (MENS), H-Wave stimulation device, electro-acuscope myopulse, microcurrent therapy (MCT), InterX 1000 neurostimulator device, electro therapeutic point stimulation (ETPS) also known as microcurrent point stimulation (MPS), scrambler therapy device, and other technology used to perform a massage function. Further, the customizable artificial hand massage apparatus may include one or more massage heads for performing the massage function.

Additionally, the massage application included as a part of the customizable artificial hand massage apparatus may be configured to detect sounds such as, for example, a baby crying and other sounds. The customizable artificial hand massage apparatus may include a microphone to detect sounds. For example, when the massage application detects a sound such as a baby crying, the massage application may cause the customizable artificial hand apparatus to automatically turn on in response to the detected sound. Additionally, the massage application may be configured to record the date and time that baby began to cry. The massage application may be configured to determine how long the baby cried or the duration of a particular sound. Alternatively, the massage application may be configured to determine that the sound has ceased and cause the customizable artificial hand massage apparatus to automatically turn off once the sound ceases or the sound falls below a predetermined sound threshold. The massage application may also be configured to record the date and time that the baby stop crying, the sound ceased, or the sound fell below a predetermined sound threshold.

The massage application component of the customizable artificial hand massage apparatus may be configured to detect various types of vibrations and various types of movements. For example, assuming the customizable artificial hand massage apparatus is placed in an infant's clothing item, if an infant wearing the clothing item begins to move around, the massage application may be configured to detect such movement and send an alert or a notification to the control application. Alternatively, the massage application may trigger an alarm such as a beeping noise, lighted indicators, or other notifications that may be present on the customizable artificial hand apparatus.

Similarly, the massage application component of the customizable artificial hand massage apparatus may also be configured to determine how far an object or person is relative to the customizable artificial hand massage apparatus. For example, the customizable artificial hand massage apparatus may configured to detect when a person comes within a predetermined distance of the customizable artificial hand massage apparatus.

The massage application component of the customizable artificial hand massage apparatus may also be configured to detect when gas is expelled from an infant and count the amount of times gas is expelled. The customizable artificial hand massage apparatus may be further configured to store information such as, for example, the amount of gas expelled from an infant. This information may be stored or transferred information electronically to the control application using the massage application.

The massage application component of the customizable artificial hand massage apparatus may also be configured to determine the number of times an infant needs to burp. For example, the massage application component of the customizable artificial hand massage apparatus may be configured to send a notification alerting a user of the amount of gas expelled from a baby and the number of times that a baby has burped. In response to the notification, the customizable artificial hand massage apparatus may be configured to automatically cease operation when desired amount of gas has been expelled or the baby has burped a specified number of times.

The customizable artificial hand massage apparatus may be configured to be monitored and controlled by a control application on a client device, a control panel positioned on the customizable artificial hand massage apparatus, a remote control device, and other control mechanisms.

The control application may be configured to receive and store information from the massage application such as, for example, sound notifications, vibration notifications, movements, and other information. The control application may be configured to communicate electronically with the massage application in order to send a notification of crying, burping sounds, vibrations, movement and the number of times gas is expelled from an infant. In the event that a component of the customizable artificial hand massage apparatus malfunctions, the massage application may be configured to detect which component is the source of the malfunction and provide an indicator either on the control panel of the customizable artificial hand massage apparatus or send a notification to the control application. In the following discussion, a general description of the system and its components is provided, followed by a discussion of the operation of the same.

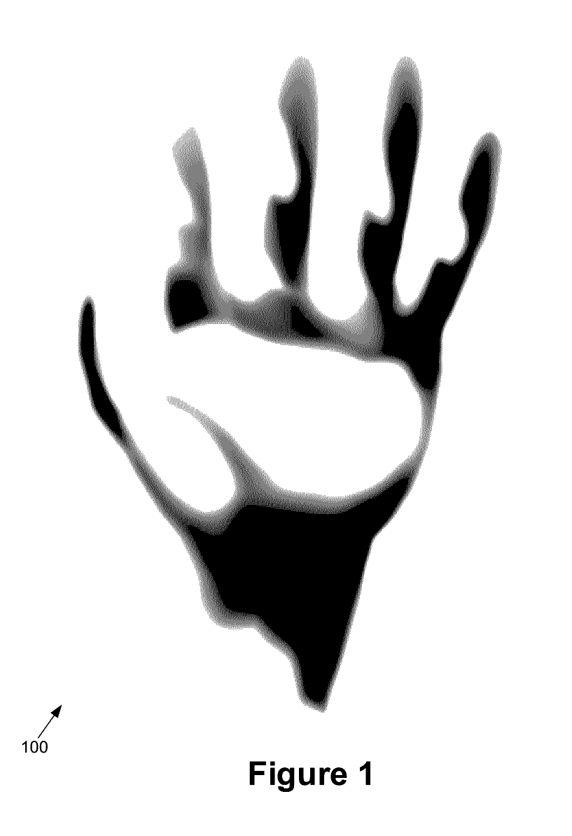

With reference to FIG. 1, shown is an example of a customizable artificial hand massage apparatus 100. The customizable artificial hand massage apparatus may operate as a stand-alone massage device. Alternatively, the customizable artificial hand massage apparatus may be configured to be inserted in a product or built in a product. The customizable artificial hand massage apparatus 100 may be configured to function equivalently to an actual human hand.

A user may insert his or her hand into a molding area associated with the customizable artificial hand apparatus and a customized artificial hand corresponding to the dimensions of the user's hand may be generated within the molding area. The molding area may be included as a part of a product such as, for example, baby carriers, mattresses, chairs, clothing items, strollers, baby swings, baby car seats, vehicle car seats, and other products. As a non-limiting example, the customizable artificial hand massage apparatus may be created using a suction mechanism configured to perform a suction action around the hand of a user. For example, when a user inserts his or her hand into the molding area, the suction mechanism may be configured to perform a suction function and generate a customized artificial hand that corresponds to the dimensions of the user's hand. The molding area may include alginate mold, gel-10, silicone or other substances that may be used for molding.

In one embodiment, the customizable artificial hand massage apparatus may be included as a part of an infant's jumpsuit. When worn by an infant, the jumpsuit would cover the infant's upper back, lower back, chest, buttocks and stomach. Nodes similar to massage nodes may be positioned in the aforementioned areas of the jumpsuit. A user may select a mode of operation which mimics the movements of an actual human hand. For example, the user may select a patting operation which simulates patting the infant on the back. The user may also select a finger tap operation which simulates finger tapping the infant on the back. Other modes may include an open hand rubbing operation which simulates a vertical, horizontal or circular rubbing motion on the back and stomach area or an open hand pat operation which simulates an open hand patting movement on the back or buttocks.

In another embodiment, the customizable artificial hand massage apparatus may be generated by performing a scan a hand of user using a scanning device to determine a plurality of dimensions associated with the hand of the user such as, for example, shape, size, weight, user specific motions, and other dimensions. Once the plurality of dimensions associated with the hand of the user has been determined by the scanning device, the scanning device may then send the plurality of dimensions associated with the hand of the user of a product in which the customizable artificial hand massage apparatus may be generated such that the customizable artificial hand corresponds to the plurality of dimensions associated with the hand of the user.

In yet another embodiment, the customizable artificial hand may be generated based at least in part on a set of images of the hand of the user such that the set of images depict a plurality of dimensions associated with the hand of the user. For example, the set of images may be two-dimensional or three-dimensional images. The set of images may be sent to a product comprising the molding area in which the customizable artificial hand massage apparatus may be generated based at least in part on the set of images.

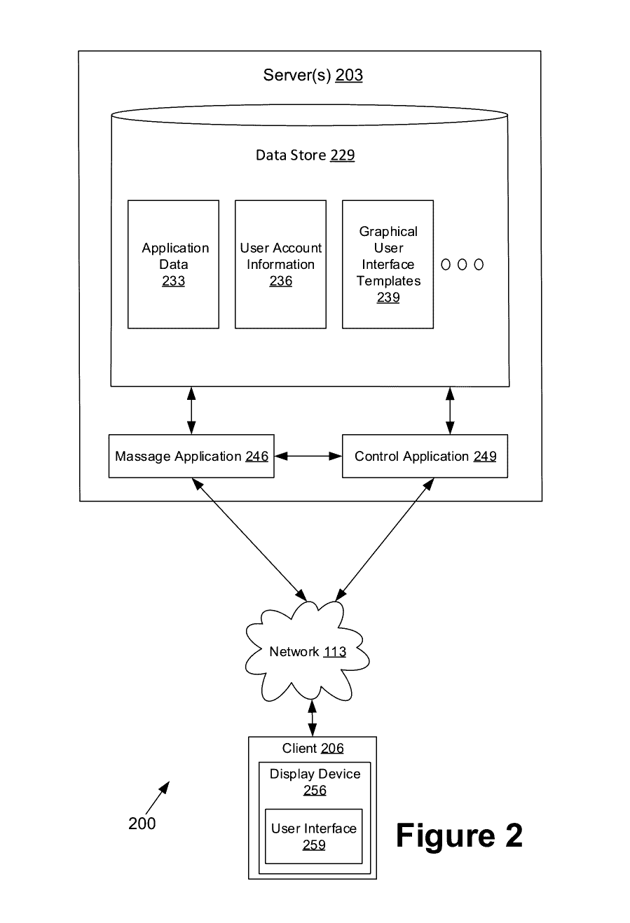

Turning now to FIG. 2, shown is a networked environment 200 that includes, for example, at least one server 203, and a-client 206. The server 203 may represent multiple servers that may be arranged to work in coordination with each other. Alternatively, such servers 203 may be arranged in some other manner, as can be appreciated. The client 206 is configured to access information on the server 203 as will be described. Both the server 203 and the client 206 are coupled to a network 213. The client 106 may comprise for example, a processor-based system such as a computer system. Such a computer system may be embodied in the form of a desktop computer, a laptop computer, a personal digital assistant, a cellular telephone, web pads, tablet computer systems, smartphones, and other devices with like capability. The network 213 may comprise, for example, any type of network environment such as the Internet, intranets, local area networks, wide area networks, wireless networks, or other networks, or a combination of two or more of such networks as can be appreciated. Although only a single client 206 is shown, the client 206 represents many clients 106 that can exist on the network 213.

According to various embodiments, the server 203 includes various applications that are executed, for example, to facilitate the detection of events, storing of information, transferring of information, monitoring and control of the customizable artificial hand massage apparatus 100 (FIG. 1).

The data in the data store 229 is used during the normal operation of the massage application 246 and the control application 249. For example, stored within the data store 129 are application data 233, user account information 236, graphical user interface templates 239, and other information as can be appreciated.

The application data 233 may include various information such as dates and times associated with events, modes of operation, settings, initial state data, predetermined threshold sound measurements, and/or other application data 233 as can be appreciated. The user account information 236 may include personal information about various users such as, for example, name, address, passwords, dimensions associated with the user's hand, one or more images corresponding to the user's hand, and other information. The graphical user interface templates 239 may include various user interface layouts and other components that are used by the control application 249 to generate user interfaces that are served up to the client 206 as will be described. In addition, there may be other information included in the data store 229 as can be appreciated.

According to various embodiments, the massage application 246 and the control application 249 may be executed by the server 203. However, it is possible that the massage application 246 and the control application 249 may employ some other technology such as, for example, a client side application, that allows the massage application 246 and the control application 249 to interface with the server 203 as can be appreciated.

The client 206 may include a display device 256 that is employed to render user interfaces 259 that may comprise a portion of a network page. Such network pages may be generated dynamically using various software platforms such as AJAX, PERL, JAVA, or other software platforms as can be appreciated.

The user of a client 206 may manipulate the respective user interfaces 259 to control and monitoring the events and activity associated with the customizable artificial hand massage apparatus 100 (FIG. 1). In addition, a user may manipulate the user interface(s) 259 rendered on the display device 256 of the client 206 to facilitate other functions as will be described. The display device 256 may be any type of display device including a liquid crystal display (LCD), a cathode-ray tube (CRT), a flat plasma panel display, or other display device.

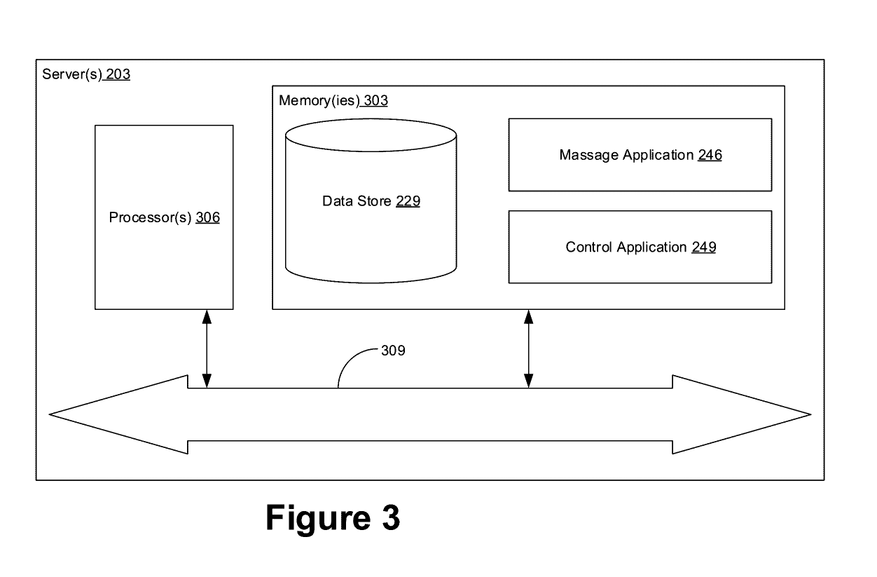

With reference to FIG. 3 shown is a schematic block diagram of the server(s) 203 according to an embodiment of the present disclosure. The server(s) 203 includes at least one processor circuit, for example, having a processor 306 and a memory 303, both of which are coupled to a local nterface 309. To this end, the server(s) 203 may comprise, for example, at least one server computer or like device. The local interface 309 may comprise, for example, a data bus with an accompanying address control bus or other bus structure as can be appreciated. Stored in the memory 303 are both data and several components that are executable by the processor 306. In particular, stored in the memory 303 and executable by the processor 306 are the massage application 246 and control application 249 and potentially other applications. Also stored in the memory 303 may be a data store 229 and other data. In addition, an operating system may be stored in the memory 303 and executable by the processor 306.

It is understood that there may be other applications that are stored in the memory 303 and are executable by the processors 306 as can be appreciated. Where any component discussed herein is implemented in the form of software, anyone of a number of programming languages may be employed such as, for example, C, C++, C#, Objective C, Java, Javascript, Perl, PHP, Visual Basic, Python, Ruby, Delphi, Flash, or other programming languages.

A number of software components are stored in the memory 303 and are executable by the processor 306. In this respect, the term “executable” means a program file that is in a form that can ultimately be run by the processor 306. Examples of executable programs may be, for example, a compiled program that can be translated into machine code in a format that can be loaded into a random access portion of the memory 303 and run by the processor 306, source code that may be expressed in proper format such as object code that is capable of being loaded into a random access portion of the memory 303 and executed by the processor 306, or source code that may be interpreted by another executable program to generate instructions in a random access portion of the memory 303 to be executed by the processor 306, etc. An executable program may be stored in any portion or component of the memory 303 including, for example, random access memory (RAM), read-only memory (ROM), hard drive, solid-state drive, USB flash drive, memory card, optical disc such as compact disc (CD) or digital versatile disc(DVD), floppy disk, magnetic tape, or other memory components.

The memory 303 is defined herein as including both volatile and nonvolatile memory and data storage components. Volatile components are those that do not retain data values upon loss of power. Nonvolatile components are those that retain data upon a loss of power. Thus, the memory 303 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 306 may represent multiple processors 306 and the memory 303 may represent multiple memories 303 that operate in parallel processing circuits, respectively. In such a case, the local interface 309 may be an appropriate network 213 (FIG. 2) that facilitates communication between any two of the multiple processors 306, between any processor 306 and any of the memories 303, or between any two of the memories 303, etc. The local interface 309 may comprise additional systems designed to coordinate this communication, including, for example, performing load balancing. The processor 306 may be of electrical or of some other available construction.

Although the massage application 246 and the control application 249, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or steady state machine that employs any one of or—a combination of a number of technologies. These technologies may include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

Also, any logic or application described herein, including the massage application 246 and the control application 249, that comprises software or code can be embodied in any non-transitory computer-readable medium for use by or in connection with an instruction execution system such as, for example, a processor 306 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer readable medium may be a read-only memory (ROM), a programmable read-only memory (PROM), an erasable programmable read-only memory (EPROM), an electrically erasable programmable read-only memory (EEPROM), or other type of memory device.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A massage apparatus comprising:
a massage structure, wherein the massage structure comprises a customizable artificial hand.

2. The massage apparatus of claim 1, wherein the customizable artificial hand being created using a suction mechanism, wherein the suction mechanism is configured to perform a suction action around a hand of a user, wherein the hand of the user being inserted into a predefined area, wherein the suction mechanism is configured to determine a plurality of dimensions associated with the hand of the user, wherein the suction mechanism is configured to halt the suction action, wherein the suction mechanism is configured to generate the simulated hand, wherein the simulated hand corresponds to the plurality of dimensions associated with the hand of the user.

3. The massage apparatus of claim 1 further comprising at least one electrical stimulation device.

4. The massage apparatus of a claim 1 further comprising:
a plurality of massage heads.

5. The massage apparatus of claim 1 further comprising a device configured to scan a hand of user to determine a plurality of dimensions associated with the hand of the user.

6. The massage apparatus of claim 5, wherein the device is further configured to send the plurality of dimensions associated with the hand of the user to a product.

7. The massage apparatus of claim 6, wherein the product is onfigured to generate the customizable artificial hand, wherein the customizable artificial hand corresponds to the plurality of dimensions associated with the hand of the user.

8. The massage apparatus of claim 1, wherein the customizable artificial hand is generated based at least in part on a set of images of the hand of the user, wherein the set of images depict the plurality of dimensions associated with the hand of the user.

9. The massage apparatus of claim 8, wherein the set of images corresponds to two-dimensional images.

10. The massage apparatus of claim 8, wherein the set of images corresponds to three-dimensional images.

11. The massage apparatus of claim 1, wherein the customizable artificial hand is configured to be placed in a product.

12. The massage apparatus of claim 1, further configured to perform a set of massage movements equivalent to that a user of the customizable artificial hand massage apparatus.

13. A method comprising the steps of:
detecting, in a computing device, an occurrence of an event associated with a user of a customizable artificial hand massage apparatus;
performing, in a computing device, a function in response to the occurrence of the event,
determining, in the computing device, an amount of tension relieved from a set of muscles associated with the user of the customizable artificial hand massage apparatus;
adjusting, in the computing device, an amount of pressure applied by the customizable artificial hand massage apparatus based at least in part on the determined amount of tension relieved from the set of muscles associated with the user of the customizable artificial hand massage apparatus;
storing, in the computing device, a set of information associated with the user of the customizable artificial hand massage apparatus; and
transferring, in the computing device, the set of information to a control application, the control application being configured to provide remote access and control to the customizable artificial hand massage apparatus.

14. The method of claim 13, further comprising the step of:
determining, in the computing device, whether a noise level is above a predetermined threshold noise level.

15. The method of claim 14, further comprising the step of:
sending, in the computing device, a request to automatically turn on or turn off the customizable artificial hand massage apparatus, based at least in part on the determined noise level.

16. The method of claim 14, further comprising the step of:
detecting, in the computing device, a type of vibration associated with the user of the customizable artificial hand massage apparatus.

17. The method of claim 16, further comprising the step of:
sending, in the computing device, a request to automatically turn on or turn off the customizable artificial hand massage apparatus, based at least in part on the type of vibration.

18. The method of claim 14, further comprising the step of:
detecting, in the computing device, one or more movements associated with the user of the customizable artificial hand massage apparatus.

19. The method of claim 16, further comprising the step of:
sending, in the computing device, a request to automatically turn on or turn off the customizable artificial hand massage apparatus, based at least in part on the one or more movements.

20. The method of claim 14, further comprising the step of:
determining, in the computing device, a number of times that an amount of gas is expelled from a user of the customizable artificial hand massage apparatus.
